(19) United States
(12) Patent Application Publication
(10) Pub. No.: US 2009/0196910 A1
(43) Pub. Date: Aug. 6, 2009

(54) Sustained-Release Preparation of Statin Drugs

(75) Inventors: Hongping Yie, Huaibei (CN); Zuolin Zhu, San Diego, CA (US); Meg M. Sun, San Diego, CA (US)

Correspondence Address: HAMRE, SCHUMANN, MUELLER & LARSON, P.C., P.O. BOX 2902, MINNEAPOLIS, MN, 55402-0902, US

(73) Assignee: PFICKER PHARMACEUTICALS LTD., Huaibei (CN)

(21) Appl. No.: 11/989,086

(22) PCT Filed: Nov. 21, 2005
(86) PCT No.: PCT/CN2005/001967

§ 371 Date: Apr. 8, 2009

(30) Foreign Application Priority Data
Jul. 19, 2005 (CN) 200510085860.0

(51) Int. Cl. A61K 9/70; A61K 31/351; A61L 15/00

(52) U.S. Cl. 424/448; 514/460; 604/307

(57) ABSTRACT

A preparation of statins for prolonged release. The preparation is a transdermal therapeutic system or hypodermic implantation system. And they overcome the problem of low bioavailability and increase patient compliance. The statins in the invention contain all of statins and responding salts, hydroxyl ester derivatives at 4-position. The invention has many advantages: for example, reduce dosage frequency, remain stable and lasting blood concentration of medicine, further improve the curative effect, provide patients with a convenient and safety means of administration, and obtain an effective period of up to seven days to months by taking medicine once.

### FIELD OF THE INVENTION

This invention relates to a novel pharmaceutical preparation, more specifically, a sustained-release preparation of statin drugs.

### BACKGROUND

Statin drugs, i.e., HMG-CoA reductase inhibitors, have been known since the end of the last century for its benefits for cardio- and cerebro-vascular diseases. The statin drugs can reduce the endogenous synthesis of cholesterols and prevent the onset and the development of atherosclerosis, and are therefore used as an effective therapy against primary hypercholesterolemia. The statin drugs, primarily known as hypolipidemic drugs, are now found also useful in the treatment of the conditions such as osteoporosis, the Alzheimer's disease, cardiac diseases, organ transplantation, stroke and diabetes.

At present, the statin drugs are most often orally administrated on a daily basis. However, several problems have been found associated with the daily oral administration. For example, after the administration, the bio-availability and the general circulation of statin drugs are fairly low due to the first-pass metabolism in the liver and the clearance by the digestive system. For instance, atovastatin reaches the plasma peak about 1-2 hours after the oral administration, providing a bio-availability no more than 14% (www.lipitor.com); simvastatin reaches the plasma peak about 4 hours after the oral administration, while the plasma lever after 12 hours is only about 10% of the peak, and only about 5% of simvastatin enters into the general circulation (www.zocor.com). Thus, increased dosages of statin drugs are usually used to obtain the expected therapeutic efficacy. Nevertheless, increasing evidences show that statin drugs may have significant side-effects on the liver, the kidney, the muscular tissue, etc., which has created a calling for a decreased dosage of statin drugs. Further, the therapeutic efficacy may also be diluted by the patient's failure in sticking with the frequent administrations.

Though studies on statin drugs have always been hot, not any sustained-release preparations of the same have been reported so far. There exists a need for a sustained-release preparation of statin drugs to resolve the problems in the prior art as said above.

### THE SUMMARY OF THE INVENTION

One object of the invention is to provide a sustained-release preparation, with which the required dosage of statin drugs is decreased, the statin-associated side-effects are reduced, and the patient's compliance is improved. The invention provides a sustained-release preparation of statins comprising a transdermal therapeutic system or a subcutaneous implantable delivery system. The other object of the invention is to provide a method for preventing the crystallization of statins in a drug reservoir layer.

1. The Statin Drugs Useful in the Invention

In the present invention, there is no limitation on the useful statin drugs. The statin drugs can be used alone or in combination in a delivery system. The useful statin drugs include but are not limited to: lovastatin, simvastatin, pravastatin, atovastatin, rosuvastatin, fluvastatin, pitavastatin and huivastatin (PCT/CN2004/001370 filed by the present inventors). Also included are the pharmaceutical salts of these drugs such as those formed with potassium, sodium and calcium, the prodrugs of the statin drugs such as the small chemical drugs described in the Chinese patent applications 2003101030307 and 2003101030311 (also filed by the present inventors), and the pharmaceutically acceptable ester derivatives of the statin drugs.

As used herein, the said ester derivatives of statin drugs refer to the esters formed at the hydroxyl group at position 4 (4-hydroxy group), which can be, for example, an acetate, a formate, a propionate or a butyrate. Particularly, a said ester derivative of lovastatin formed at the 4-hydroxy group may have the following formula:

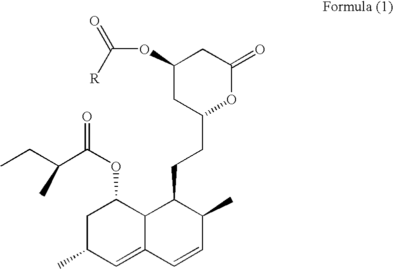

wherein, R=hydrogen, methyl, ethyl or propyl.

As shown in the in vitro experiments, the esters of statin drugs formed at the 4-hydroxyl group can be transformed into statin drugs by many dominant human ester hydrolases at a pH of 6.5-7.5. For example, acetate of simvastatin formed at position 4 can be readily transformed into simvastatin by carboxylate hydrolases in human blood. At pH 7.4, the rate of hydrolysis of 4-acetate of simvastatin is about 8.0 mmol/mg enzyme/hour (3.3 g/mg enzyme/hour).

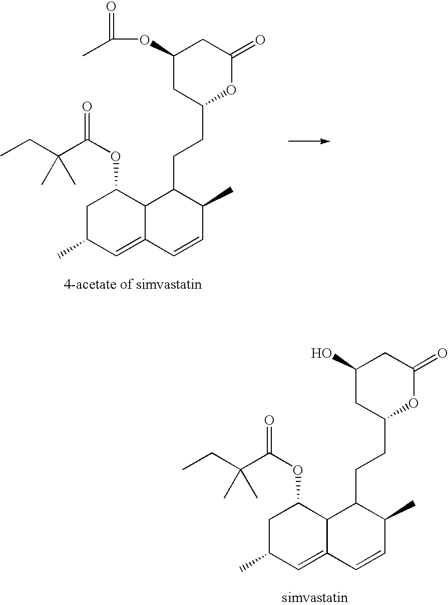

The carboxylate hydrolases can be isolated from the plasma and the method for isolating carboxylate hydrolases and the method for determining the hydrolysis rate of 4-acetate of simvastatin have been reported in the literatures (see, for example, The Journal of Biological Chemistry, US, 1985, 260, 5225).

2. Transdermal Therapeutic System

As used herein, the term “transdermal therapeutic system (TTS)”, which may be used as exchangeable with the term “transdermal delivery system”, refers to a controlled-release preparation providing a systematic therapeutic effect by delivering drugs though the skin. TTS is advantageous over other routes of administration in evading the “first-pass metabolism” by the liver, exempt from the impacts of the gastrointestinal fluids, providing controllable sustained effects, reducing toxicity and side-effects, providing a sustained and stable blood level, which finally enhance the therapeutic effect, allow decreased dosing frequency and ease the administration.

The transdermal therapeutic system according to the present invention may be in various forms including but not limited to:

(1) An adhesive device, such as a conventional simple adhesive patch comprising a statin drug(s)-containing polymer pressure-sensitive adhesive layer and a vapor permeability and water resistant backing layer;

(2) A monolithic device, such as a monolithic patch comprising a pressure-sensitive adhesive layer, a statin drug(s)-containing release rate-controlling polymeric matrix layer and a vapor permeability and water resistant backing layer;

(3) A reservoir device, such as a reservoir-typed adhesive patch comprising a pressure-sensitive adhesive layer, a release rate-controlling membrane, a statin drug(s)-containing polymeric reservoir layer and a vapor permeability and water resistant backing layer;

(4) An iontophoretic delivery patch containing salts of statin drugs such as those formed with potassium, sodium and calcium. The iontophoretic delivery patch can be prepared as previously taught in, for example, US2004/0077991 A1, US2004/0039328 A1 and US2004/0225253 A1.

The sustained-release preparation of statin drugs of the invention is further described in details as follows.

The preparation of the invention may comprise a pressure-sensitive adhesive layer containing or not containing a statin drug. The pressure-sensitive adhesive layer is made of a polymeric material that is selected to be capable of dispersing or releasing the statin drugs while not being irritating to the skin or undesirably reacting with the drugs to cause their degeneration. The pressure-sensitive adhesive layer should be capable of providing sufficient and durable adhesion to allow a prolonged use, while not to cause lesions to the skins when being peeled off. The pressure-sensitive adhesive layer can be a single layer or a multiple-layered laminate. Usually, the pressure-sensitive adhesive layer has a glass transition temperature (TG), which can be determined by a Differential Scanning Calorimeter (DSC) in the range of −70° C. to 0° C.

The suitable polymers for the pressure-sensitive adhesive layer include but are not limited to, for example, acrylate polymers, silicon polymers and rubber polymers.

Acrylate polymer-based pressure-sensitive adhesive materials include, for example, methacrylic acid polymers, butyl acrylate polymers, butyl methacrylate polymers, hexyl acrylate polymers, hexyl methacrylate polymers, 2-ethylbutyl acrylate polymers, 2-ethylbutyl methacrylate polymers, isooctyl acrylate polymers, isooctyl methacrylate polymers, 2-ethylhexyl acrylate polymers, 2-ethylhexyl methacrylate polymers, decyl acrylate polymers, decyl methacrylate polymers, dodecyl acrylate polymers, dodecyl methacrylate polymers and combinations thereof. Commercially available polyacrylic materials include, for example, the Duro-Taks (e.g., Duro-Tak 87-2194, Duro-Tak 87-2196, Duro-Tak 87-1197, Duro-Tak 87-4194, Duro-Tak 87-2510, Duro-Tak 87-2097 and Duro-Tak 87-2852) from the National Starch and Chemical (Bridgewater, N.J. USA) and the GELVA-Multipolymer Solutions (GMSs) (e.g., GMS737, GMS788, GMS1151, GMS3087 and GMS7882) from the Monsanto Company (St. Louis, Mo. USA).

The silicon polymer-based pressure-sensitive adhesive materials include, for example, the polymeric materials listed in the Handbook of Pressure-Sensitive Adhesive Technology (Sobieski, the 2nd ed., 508-517, “Silicone Pressure Sensitive Adhesives”, N.Y., 1989). The commercially available silicon polymer-based pressure-sensitive adhesive materials include, for example, the BIO-PSA 7-4503, BIO-PSA 7-4603, BIO-PSA 7-4301, BIO-PSA 7-4202, BIO-PSA 7-4102, BIO-PSA 7-4106, BIO-PSA 7-4303, etc. from the Dow Corning (Midland, Mich. USA).

Rubber-based pressure-sensitive adhesive materials may comprise a combination of polymeric materials of different kind or polymeric materials of different molecular weights. The specific examples include but are not limited to: polyisobutylene rubber, natural and synthetic polyisoprene rubbers, polybutylene- and polyisobutylene-rubbers, styrene-butadiene block copolymer-rubber, styrene-isoprene-styrene block copolymer-rubber, butyl rubber, polytetrafluoroethylene rubber, polyvinylchloride rubber, polyvinylidene chloride rubber, polychlorodiene rubber, and co-polymers thereof.

When the pressure-sensitive adhesive layer contains one or more statin drugs as the active ingredient(s), the statin drug(s) will be present in an amount of 0.1 wt %—10 wt %. Optionally, the said adhesive layer may further contain one or more additives such as a skin-penetration enhancer, a crystallization inhibitor to prevent the statins from crystallizing, an antioxidant, an age-protecting agent, a plasticizer and a tackifying agent to improve the adhesion of the said pressure-sensitive adhesive layer, an anti-infectious antiphlogistic in appropriate amounts.

According to the present invention, the release rate-controlling membrane may be a dense membrane, which is permeable to statin drugs and additional adjuvant agents, or a microporous material, which allows the statin drugs and additional adjuvant agents to penetrate through micropores. The release rate-controlling membrane is designed to be capable of delivering about 5-40 mg/day of the statin drug(s) from a patch to the skin surface at a constant rate, wherein the patch is designed to be capable of reserving the statin drugs in an amount sufficient for 7 to 10 days of delivery. Preferably, the release rate-controlling membrane is made from polysiloxanes, especially poly(dimethyl siloxane) (PDMS), wherein, a polyethylene oxide (PEO) may optionally be added to increase the penetration rate of the statins such as pravastatin or to decrease the penetration rate of, e.g., simvastatin.

According to the present invention, the statin drug(s)-containing polymeric drug reservoir layer may comprise a polymeric material and a statin drug as the major components, and optionally comprise additional agents such as a skin-penetration enhancer, a crystallization inhibitor to prevent the statins from crystallizing, an antioxidant, an age-protecting agents, a preservative, an anti-infectious antiphlogistic. In some embodiments, an adhesion enhancer such as a plasticizer and/or a tackifying agent may also be included. The said polymeric material may be wool wax, or be the same as the polymeric material for the pressure-sensitive adhesive layer, which include, for example, polyacrylate polymers, silicon polymers, rubbers, etc., as said above.

The statin drug(s) may be present in the polymeric drug reservoir layer at a weight percentage of about 6%—90%, more usually, 10 wt %—50 wt %. The polymeric materials should be selected to be capable of dispersing or releasing the statin drug while not undesirably reacting with the drugs to cause degeneration.

According to the present invention, suitable skin-penetration enhancers include, for example, anionic surfactant, cationic surfactant and nonionic surfactant. The specific examples include but are not limited to: Azone, propylene glycol (PG), oleic acid (OA), linoleic acid, dodecyl N, N-dimethylaminoisopropionate, dodecyl N, N-dimethylaminoacetate, sorbitan sesquioleate, cetostearyl alcohol, polysorbate 60, sorbitan monostearate, vegetable oils and vegetable alcohols such as mentha-camphor, menthol, mint oil, and combinations thereof. The skin-penetration enhancer may be a multiple-component system, such as the Azone-propylene glycol-based and the oleic acid-based binary systems.

Optionally, a cosolvent may be included to improve the solubility of the statin drugs in the polymeric drug reservoir layer. The suitable cosolvents include but are not limited to: lecithin, retinal derivatives, tocopherol, dipropylene glycol, triacetin, propylene glycol, saturated and unsaturated aliphatic acids and mineral oils such as liquid paraffin.

According to the present invention, the vapor permeability and water resistant backing layer may consist of a single layer or a multiplicity of layers. The suitable materials for the said backing layer include but are not limited to: woven fabrics, non-woven fabrics and resin films. The suitable resin films include but are not limited to those made from polyurethane, polyethylene, silicone resins, natural and synthetic rubbers, polyglycolic acid, polylactic acid, polyvinyl alcohol, polyvinylpyrrolidone, collagen, gelatin, hyaluronic Acid, sodium Alginate, chitin, chitosan, fibrin and cellulose. The material(s) and the structure of the backing layer should be appropriately selected and designed so as to make the backing layer permeable to vapor while impermeable to fluid.

In most of the cases, the transdermal therapy patch system of the invention comprises a peelable release liner. The peelable release liner is laid on one side of the pressure-sensitive adhesive layer, and is to be peeled off before use. The suitable materials for the peelable release liner include but are not limited to: paper, siliconized polyester films and plastic films.

In a second aspect, the present invention provides a subcutaneous implantable sustained-release system. This system is most preferred for the medication against chronic diseases because it minimizes the patients' incompliance. In one of the embodiments, the said subcutaneous system may be implanted via injection of erodible polymeric drug-depot, which provides a sufficient dosage of weeks. In a further embodiment, the said subcutaneous implantable sustained-release system is a device capable of reserving a dosage sufficient for use over quite a period of time, e.g., as long as 12 months.

The subcutaneous implantable sustained-release system of the invention comprises an outside surface as a first component and a statin drug(s)-containing core as a second component. The said outside surface may be composed of a non-degradable polymeric film such as ethylene-vinylacetate copolymer, standard stainless steel sheet or titanium alloy sheet or minitube. A suitable release system includes a release rate-controlling membrane, which may be of the same as said above for the release rate-controlling membrane of the patch. The drug-containing core comprises at least one statin drug as the effective ingredient and a filler such as cyclodextrin, hydrogel and/or other polymeric materials. Optionally, additives such as a crystallization inhibitor to prevent statins from crystallizing, an antioxidant, an age-protecting agent, a cosolvent, a preservative and other adjuvants that help in stabilizing the statin drugs may also be included as desired. In this context, the terms “polymeric material” may be the ethylene-vinylacetate copolymer or the polymers used in the polymeric drug reservoir layer as said above.

The subcutaneous implantable delivery system may be in various shapes such as a cylinder, a cubic, a rectangular cubic or a sphere. The said system comprises a stantin drug(s)-containing polymeric core and an outside coating film. The said outside coating film may be either permeable or impermeable to the drug. For a drug permeable film, suitable micorpores are usually provided to allow drug penetration.

In a third aspect, the present invention provides a method for preventing crystallization of statin drugs.

For the two sustained-release preparations as described above, since the medication of statin drugs always continues for years, it would be desirable to maximize the concentration of the drug as appropriate in a delivery system. When being delivered at a lever of saturation or even supersaturation, statin drugs incline to precipitate as crystals. For transdermal absorption and for a subcutaneous implantable delivery system, the crystallization and precipitation of the active ingredient(s) is undesirable for delivering them into the body. Particularly, for a transdermal therapeutic system, the crystallization and precipitation may also cause irritation to the skin, which may undesirably affect the therapeutic efficacy.

The present invention successfully resolves the problem of statin crystallization in the drug reservoir in transdermal therapeutic system and subcutaneous implantable delivery system.

The present inventors find that transforming the statin drugs into their ester derivatives can significantly decrease the crystallization in the drug reservoir in transdermal therapeutic system and subcutaneous implantable delivery system. Some of the said derivatives, such as the esters derivatives of simvastatin as shown below, are naturally present as a liquid at the room temperature.

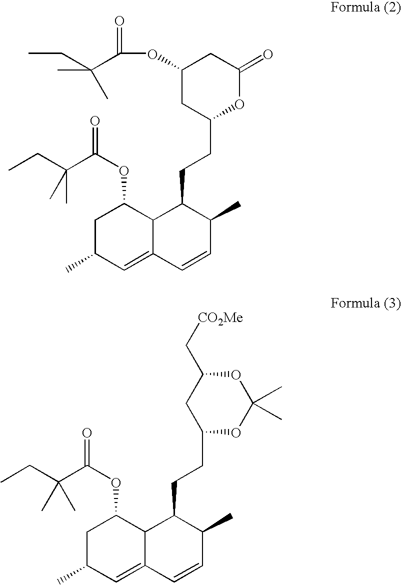

In some embodiments, the method of the invention prevents the statin crystallization by forming derivatives such as a 4-carboxylate of simvastatin of formula (2) and the derivative of formula (3). In the other embodiments, the method of the invention also uses one or more inhibitors of statin crystallization such as polyvinylpyrrolidones (PVPs), which includes homopolymers such as povidone and polyvidone and block co-polymers such as those containing vinyl acetate units. The commercially available PVPs include, for example, those available from the BASF AG (Ludwigshafen, Germany) in the name of Kollidon, such as Kollidon 10, Kollidon 17 PF, Kollidon 25, Kollidon 90, Kollidon 30 and VA 64, and the like.

The present inventors also find that when the derivatives as the side-products resulting from the dehydroxylation of statin drugs are present at a level no less than 1.0%, the crystallization in the reservoir is significantly decreased. For example, when the side-products of formula (4) resulting from the dehydroxylation of simvastatin are present in the raw statin drugs at a level of 1.0%, in a reservoir containing simvastatin in an amount of 10.0% (by weight), no detectable crystallization is observed. Normally, the presence of these dehydroxylation side-products in the statin drugs is allowable as long as the total impurity is not more than, e.g., 2.0% (e.g., for simvastatin).

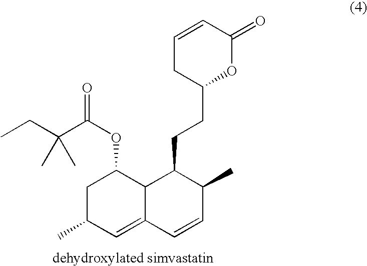

Further, the present inventors find that when the content of statin drug(s) in the drug reservoir approaches 10% (by weight), in the absence of inhibitors of statin crystallization, the statin drug(s) incline(s) to crystallize at the cutting edges of the transdermal therapeutic system. For this problem, the present invention provides a resolution that comprises the steps of: fabricating a patch web with a statin-free polymeric materials (such as those used in the reservoir, see above), leaving thereon circular blank areas, depositing statin-containing polymeric drug reservoirs inside of the said circular blanks, and cutting the web into individual finished patches along cutting lines in the statin-free areas.

Administration of the Systems of the Invention

One patch a time, the patch system of the invention may be topically applied to a site on the surface of the skin that is not frequently subject to abrasion. The site may be, for example, the skin area at the ear rear where is hair-free, on the arm, the leg and the abdomen. Before the application, the target site may be cleansed with water or alcohol and air died. Then, the peelable release liner is removed, and the patch is pressed onto the treated area. The patch may be replaced after 7 days with a new patch at the same site or at a different site.

The implantation of the subcutaneous implantable delivery system should be performed at a credited institute by a qualified physician. The reservoir with appropriate amount of drug(s) and the site of implantation should be determined by the physician on consideration of the particular condition of the individual recipient.

The sustained-release preparations of statin drugs of the invention significantly decrease the dosing frequency, provide sustained and stable blood level of the drug, enhance the therapeutic efficacy and improve the compliance and safety. Both preparations of the invention can provide via single dose an effective duration as long as 7 days to several months.

### DESCRIPTION OF THE FIGURES

FIG. 1 graphically shows a laminate structure of an illustrative reservoir adhesive-typed patch (a reservoir device).

FIG. 2 graphically shows a cross-sectional view of an illustrative subcutaneous implantable delivery system (not in real scale).

FIG. 3 graphically shows a method which prevents the crystallization of statin drugs at the cutting edges of a transdermal therapeutic system.

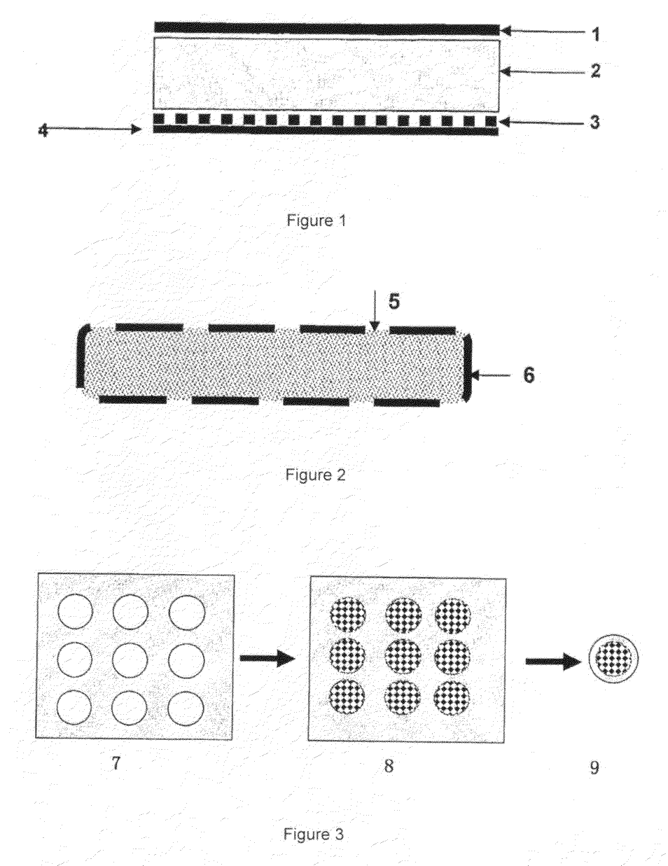

The references in the figures are defined as follows.

1—a vapor permeability and water resistant backing layer, 2—a statin drug(s)-containing polymeric drug reservoir layer, 3—a release rate-controlling membrane, 4—a pressure-sensitive adhesive layer; 5—a micropore for drug penetration, 6—a coating film; 7—a patch web with blank areas, 8—a patch web bearing statin drug(s)-containing polymeric drug reservoirs, and 9—an individual finished patch.

### Preferred Embodiments of the Invention

The invention may be better understood from the following illustrative examples. The examples are provided only for the purpose of illustration rather than limitation. In the following, the content is always determined on a dry weight basis, unless particularly specified.

### Example 1

### Preparation of a Transdermal Therapeutic System and a Subcutaneous Implantable Delivery System

1. Under a nitrogen blanket and at the room temperature, 430 g of ethanol and 215 g of ethyl acetate were added into a solution containing 1960 g of the polyacrylic polymer (Durotak 387-2287, 1004 g of solids), and were agitated to obtain a homogeneous mixture.

2. The statin drugs, the crystallization inhibitors and the antioxidant were added, as shown in Table I, into the obtained mixture. For a transdermal therapeutic system, an adhesion enhancer and a skin-penetration enhancer were also added. For a subcutaneous implantable delivery system, a hydrogel was added. The mixture was agitated to homogeneous, and then sealed in a barrel to prevent volatilization of the solvents.

TABLE I

| | | | | Subcutaneous |
|---|---|---|---|---|
| | Transdermal Delivery | | | Implant |
| Ingredients | Sample 1-1 | Sample 1-2 | Sample 1-3 | Sample 1-4 |
| Statin Drug | Simvastatin | Formula (2) | simvastatin | simvastatin |
| | 10% | 10% | 10% | 30% |
| Dehydroxylated | 0.1% | | | |
| Simvastatin | | | | |
| Polyvinylpyrrolidone | | | 10% | 10% |
| Durotak 387-2287 | 49.4% | 49.5% | 39.5% | 33% |
| methylbutylphenol | 0.5% | 0.5% | 0.5% | 0.5% |
| Wool Wax | 20% | 20% | 20% | |
| Azone | 10% | 10% | 10% | |
| Propylene glycol | 10% | 10% | 10% | |
| Tocopherol | | | | 11.5% |
| Hydrogel | | | | 15% |

In sample 1-1 and sample 1-3, no crystallization of simvastatin was observed in the obtained statin drug-containing polymeric pressure-sensitive adhesive layer after being stored under 40° C. for 10 days.

3. Production of the Preparation:

(1) For a simple adhesive patch, the statin drug-containing polymeric pressure-sensitive adhesive coating solution obtained in step 2 was applied onto a vapor permeability and water resistant backing web. The coating layer was dried via infrared radiation or hot air circulation. The web was then cut into individual patches of desired shape and size, each comprising the statin drug in a mount from 70 mg to 0.4 g. The dried pressure-sensitive adhesive layer had a thickness of about 30 μm to 3.0 mm.

(2) For a monolithic adhesive patch, the statin drug-containing polymeric pressure-sensitive adhesive coating solution obtained in step 2 was applied onto a poly(dimethyl siloxane) film as the release rate-controlling polymeric matrix layer. The coating was dried via infrared radiation or hot air circulation. Then, a vapor permeability and water resistant backing web was applied on the exposed surface of the pressure-sensitive adhesive layer. The web was then cut into individual patches of desired shape and size, each comprising the statin drug in a mount from 70 mg to 0.4 g. The dried pressure-sensitive adhesive layer had a thickness of about 30 μm to 3.0 mm.

(3) For a reservoir-typed adhesive patch, the statin drug-containing polymeric pressure-sensitive adhesive coating solution obtained in step 2 was applied onto a poly(dimethyl siloxane) film as the release rate-controlling polymeric matrix layer. The coating was dried via infrared radiation or hot air circulation.

Another pressure-sensitive adhesive coating solution was prepared according to step 2 except that the statin drug was not included. The coating solution was pre-dried into an adhesive layer. This adhesive layer optionally comprised the adhesion enhancer at a higher lever than the polymeric drug reservoir layer. Then, the adhesive layer was applied onto the exposed surface of the release rate-controlling matrix layer. Then, a vapor permeability and water resistant backing web was applied on the exposed surface of the statin-containing polymeric drug reservoir pressure-sensitive adhesive layer. The web was then cut into individual patches of desired shape and size, each comprising the statin drug in a mount from 70 mg to 0.4 g. The dried statin-containing polymeric drug reservoir pressure-sensitive adhesive layer had a thickness of about 30 μm to 3.0 mm.

(4) For a subcutaneous implantable delivery system, the statin drug-containing polymeric pressure-sensitive adhesive coating solution obtained in step 2 was filled into a syringe of a diameter of 2.55-8.38 mm. The coating solution was properly dried via infrared radiation or hot air circulation within the syringe, and was then extruded as a shaped bar. The obtained bar was cut into rods with a longitudinal dimension of 4.0 cm. Ethylene-vinylacetate copolymer minitubes (EVA minitubes, wall thickness: 0.14-0.17 mm) having a length of 5.0 cm and a diameter to match the obtained rods were provided. For example, when the rod had a diameter of 2.55 mm, the diameter of the EVA minitubes was also 2.55 mm. The EVA minitubes were soaked in dichloromethane for 1 minute. The statin drug-containing rods were inserted into the EVA minitubes. The EVA minitubes with the insertions were placed under a slow air flow at room temperature over night. Then, both ends of the minitubes were sealed at 70° C.

### Example 2

### In Vitro Drug Penetration Test

The in vitro drug penetration was determined using human skin. The skin was clamped over a Franz cell. A monolithic adhesive patch (4.8 cm², with a 1.0 mm thick drug reservoir comprising simvastatin) was applied onto the skin. The drug penetration was determined at 37° C. A 1.0% aqueous NaCl solution was used as the recipient medium. The cumulative penetration was determined as routine. The results were shown in Table II.

TABLE II

| | Time (Hour) | | | | | | | | |
|---|---|---|---|---|---|---|---|---|---|
| | 0 | 4 | 8 | 12 | 24 | 48 | 72 | 96 | 168 |
| Cumulative | 0.0 | 1.6 | 2.9 | 4.5 | 8.1 | 13.7 | 21.1 | 28.6 | 39.3 |
| Penetration | | | | | | | | | |
| (mg) | | | | | | | | | |

### Example 3

### Synthesis of the Derivative of Simvastatin of Formula (2)

Under a nitrogen blanket, 16.0 g of dry simvastatin was suspended in 300 ml of dichloromethane. The white solids dissolved quickly to give a clear solution. The solution was cooled to 5-10° C., into which 0.5 molar eq. of LiBr, 1.3 molar eq. of triethylamine and 1.4 molar eq. of 2, 2-dimethyl-butyryl chloride were added. The reaction mixture was agitated under the nitrogen blanket for 0.5 to 1 hour, and then reacted under the room temperature with continuous agitation. At the end of reaction, 100 ml water was added, and the mixture was agitated for additional 30 minutes to separate the organic phase. The obtained organic phase was sequentially washed with saturated brine (100 ml×1), saturated aqueous sodium bicarbonate solution (100 ml×4) and saturated brine (100 ml×2), and then dried over sodium sulfate. The derivative of simvastatin of formula (2) was obtained after filtration and evaporation to remove the solvents.

Melting Point (m.p.): 6.2-6.6° C.

1H-NMR (δ, CDCl3): 5.93 (d, 1H), 5.71 (dd, 1H), 5.44 (br, 1H), 5.29 (m, 1H), 5.18 (m, 1H), 4.38 (m, 1H), 2.68 (m, 3H), 2.17-2.41 (m, 4H), 1.32-1.98 (m, 11H), 1.09 (br, 12H), 1.06 (d, 3H), 0.84 (d, 3H), 0.78 (m, 6H).

### Example 4

### Synthesis of the Derivative of Simvastatin of Formula (3)

Under a nitrogen blanket, 10.0 g of dry simvastatin was dissolved in 100 ml of dichloromethane. The solution was cooled to 5-10° C., into which 10 molar eq. of dimethoxypropane and 0.4 g of paratoluenesulfonic acid were added. The reaction mixture was agitated under the room temperature for 1 hour before 3 g of sodium bicarbonate was added, and then the agitation was continued for additional 30 minutes. At the end of reaction, 100 ml water was added, and the mixture was agitated for 30 minutes to separate the organic phase. The obtained organic phase was sequentially washed with saturated brine (100 ml×1), saturated aqueous sodium carbonate solution (100 ml×3) and saturated brine (100 ml×2), and then dried over sodium sulfate. The derivative of simvastatin of formula (3) was obtained after filtration and evaporation to remove the solvents.

Melting Point (m.p.): 4.7-5.1° C.

1H-NMR (δ, CDCl3): 5.99 (d, 1H), 5.78 (dd, 1H), 5.54 (br, 1H), 5.33 (m, 1H), 4.29 (m, 1H), 3.71 (br, 1H), 3.65 (s, 3H), 1.8-2.6 (m, 5H), 1.45 (s, 3H), 1.35 (s, 3H), 1.1-1.7 (m, 11H), 1.12 (s, 3H), 1.11 (s, 3H), 1.08 (d, 3H), 0.90 (d, 3H), 0.88 (t, 3H).

### Example 5

### A Method to Prevent Statin from Crystallization at the Cutting Edges of the Transdermal Therapeutic System

As shown in FIG. 3, first, a patch web 7 was fabricated with a polymeric material not containing statin drugs, leaving thereon circular blank areas;

Second, polymeric reservoirs containing statin drug were deposited within the circular blank areas to give an adhesive patch web 8 with at least one statin drug-containing polymeric reservoirs;

Third, the patch web 8 was cut alone cutting lines in the drug-free areas into individual finished patches 9.

1. A sustained-release preparation of statin drug comprising a transdermal therapeutic system, wherein, the said transdermal therapeutic system is a simple adhesive patch system comprising a statin drug(s)-containing polymeric pressure-sensitive adhesive layer and a vapor permeability and water resistant backing layer.

2. A sustained-release preparation of statin drugs comprising a transdermal therapeutic system, wherein, the said transdermal therapeutic system is a monolithic patch system comprising a pressure-sensitive adhesive layer, a statin drug(s)-containing polymeric release rate-controlling matrix layer and a vapor permeability and water resistant backing.

3. A sustained-release preparation of statin drugs comprising a transdermal therapeutic system, wherein, the said transdermal therapeutic system is a reservoir-typed adhesive patch system comprising a pressure-sensitive adhesive layer, a release rate-controlling membrane, a statin drug(s)-containing polymeric drug reservoir layer and a vapor permeability and water resistant backing layer.

4. The sustained-release preparation according to claim 3, wherein the said pressure-sensitive adhesive layer comprises polyacrylic materials, the said release rate-controlling membrane is a poly(dimethyl siloxane) film, and the statin drug(s)-containing polymeric drug reservoir layer comprises wool wax and statin drug(s).

5. A sustained-release preparation of statin drugs comprising a subcutaneous implantable delivery system, which comprises a statin drug(s)-containing polymeric drug reservoir layer, at least one micropore for drug penetration and a coating film.

6. The transdermal therapeutic system according to claim 1, wherein, the said statin drug is selected from the group consisting of lovastatin, simvastatin, pravastatin, atovastatin, rosuvastatin, fluvastatin, pitavastatin, huivastatin, a pharmaceutically acceptable salt derivative thereof formed with potassium, sodium and calcium, and a pharmaceutically acceptable ester derivative thereof formed at the hydroxyl group at position 4 or a combination thereof.

7. The subcutaneous implantable delivery system according to claim 5, wherein, the said statin drug(s) is (are) present in the said drug reservoir at a weight percentage of 6%—90%.

8. The subcutaneous implantable delivery system according to claim 5, wherein, the said statin drug(s) is (are) present in the said drug reservoir at a weight percentage of 10%—50%.

9. A method for preventing statin from crystallization in a drug reservoir of subcutaneous implantable delivery system, which utilizes a prodrug of a statin drug, one or more polyvinylpyrrolidones or a derivative as a side-product resulting from dehydoxylation of a statin drug, wherein, the said prodrug is selected from the group consisting of ester derivatives of formula (1) and (2) formed at the hydroxyl group at position 4 and the ester derivative of formula (3) wherein the β,δ-dihydoxyl groups participate in a six-member cycloketal:

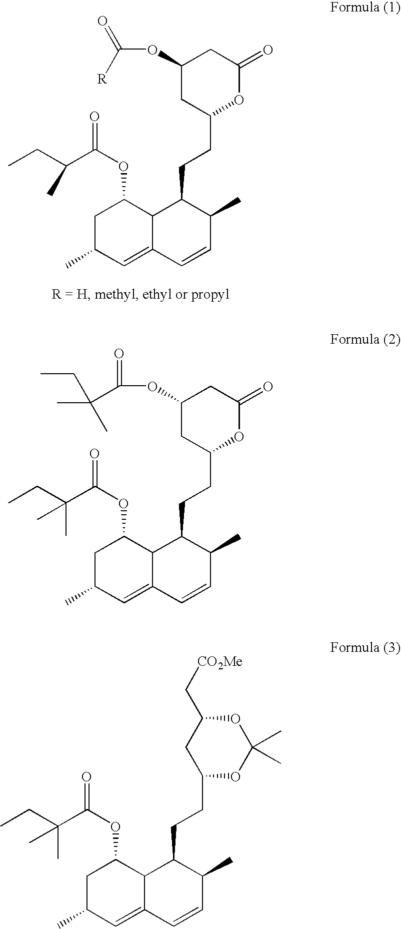

10. A method for preventing statin crystallization at the cutting edges of a transdermal therapeutic system patch, which comprises the steps of:
(1) Fabricating a patch web with a polymeric material, leaving thereon at least one circular blank area without statin drugs;
(2) Depositing a polymeric reservoir containing statin drug(s) within the individual circular blank areas to give an adhesive patch web with at least one statin drug-containing polymeric reservoir;
(3) Cutting the patch web in the drug-free areas into at least one individual patch.

11. The subcutaneous implantable delivery system according to claim 5, wherein, the said statin drug is selected from the group consisting of lovastatin, simvastatin, pravastatin, atovastatin, rosuvastatin, fluvastatin, pitavastatin, huivastatin, a pharmaceutically acceptable salt derivative thereof formed with potassium, sodium and calcium, and a pharmaceutically acceptable ester derivative thereof formed at the hydroxyl group at position 4 or a combination thereof.
